(19) United States
(12) Patent Application Publication
(10) Pub. No.: US 2006/0085081 A1
(43) Pub. Date: Apr. 20, 2006

(54) Implants and methods for treating bone

(76) Inventors: John H. Shadduck, Tiburon, CA (US); Csaba Truckai, Saratoga, CA (US)

Correspondence Address: John H. Shadduck, 350 Sharon Park Drive #B23, Menlo Park, CA, 94025, US

(21) Appl. No.: 11/146,891

(22) Filed: Jun. 7, 2005

Related U.S. Application Data
(60) Provisional application No. 60/577,562, filed on Jun. 7, 2004.

(51) Int. Cl. A61F 2/02

(52) U.S. Cl. 623/23.76

(57) ABSTRACT

An implant system that includes small cross-section implant elements that can be introduced into targeted bone regions wherein the elements self-assemble into a large cross-section, higher modulus monolith. The implant elements are configured with properties engage one another, such as surface features or magnetic properties. The implants and methods can be used to treat bone abnormalities such as compression fractures of vertebrae, bone necrosis, bone tumors, cysts and the like.

### CROSS-REFERENCE TO RELATED APPLICATIONS

This application claims benefit of Provisional U.S. Patent Application Ser. No. 60/577,562 filed Jun. 7, 2004 (Docket No. S-7700-020) titled Implant Scaffolds and Methods for Treating Tissue, the entire contents of which are hereby incorporated by reference in their entirety and should be considered a part of this specification.

### BACKGROUND OF THE INVENTION

1. Field of the Invention

This invention relates to implantable materials configured as bone support implants for treating abnormalities in bones such as compression fractures of vertebra, necrosis of femurs and the like. More in particular, the invention relates to systems for introducing small cross-section elements through a small diameter introducer wherein the elements assemble in-situ into a monolithic implant to provide bone support.

2. Description of the Related Art

Osteoporotic fractures are prevalent in the elderly, with an annual estimate of 1.5 million fractures in the United States alone. These include 750,000 vertebral compression fractures (VCFs) and 250,000 hip fractures. The annual cost of osteoporotic fractures in the United States has been estimated at $13.8 billion. The prevalence of VCFs in women age 50 and older has been estimated at 26%. The prevalence increases with age, reaching 40% among 80-year-old women. Medical advances aimed at slowing or arresting bone loss from aging have not provided solutions to this problem. Further, the affected population will grow steadily as life expectancy increases. Osteoporosis affects the entire skeleton but most commonly causes fractures in the spine and hip. Spinal or vertebral fractures also have serious consequences, with patients suffering from loss of height, deformity and persistent pain which can significantly impair mobility and quality of life. Fracture pain usually lasts 4 to 6 weeks, with intense pain at the fracture site. Chronic pain often occurs when one level is greatly collapsed or multiple levels are collapsed.

Postmenopausal women are predisposed to fractures, such as in the vertebrae, due to a decrease in bone mineral density that accompanies postmenopausal osteoporosis. Osteoporosis is a pathologic state that literally means “porous bones”. Skeletal bones are made up of a thick cortical shell and a strong inner meshwork, or cancellous bone, of collagen, calcium salts and other minerals. Cancellous bone is similar to a honeycomb, with blood vessels and bone marrow in the spaces. Osteoporosis describes a condition of decreased bone mass that leads to fragile bones which are at an increased risk for fractures. In an osteoporosic bone, the sponge-like cancellous bone has pores or voids that increase in dimension, making the bone very fragile. In young, healthy bone tissue, bone breakdown occurs continually as the result of osteoclast activity, but the breakdown is balanced by new bone formation by osteoblasts. In an elderly patient, bone resorption can surpass bone formation thus resulting in deterioration of bone density. Osteoporosis occurs largely without symptoms until a fracture occurs.

Vertebroplasty and kyphoplasty are recently developed techniques for treating vertebral compression fractures. Percutaneous vertebroplasty was first reported by a French group in 1987 for the treatment of painful hemangiomas. In the 1990's, percutaneous vertebroplasty was extended to indications including osteoporotic vertebral compression fractures, traumatic compression fractures, and painful vertebral metastasis. In one percutaneous vertebroplasty technique, bone cement such as PMMA (polymethylmethacrylate) is percutaneously injected into a fractured vertebral body via a trocar and cannula system. The targeted vertebrae are identified under fluoroscopy. A needle is introduced into the vertebral body under fluoroscopic control to allow direct visualization. A transpedicular (through the pedicle of the vertebrae) approach is typically bilateral but can be done unilaterally. The bilateral transpedicular approach is typically used because inadequate PMMA infill is achieved with a unilateral approach.

In a bilateral approach, approximately 1 to 4 ml of PMMA are injected on each side of the vertebra. Since the PMMA needs to be forced into cancellous bone, the technique requires high pressures and fairly low viscosity cement. Since the cortical bone of the targeted vertebra may have a recent fracture, there is the potential of PMMA leakage. The PMMA cement contains radiopaque materials so that when injected under live fluoroscopy, cement localization and leakage can be observed. The visualization of PMMA injection and extravasion are critical to the technique and the physician terminates PMMA injection when leakage is evident. The cement is injected using small syringe-like injectors to allow the physician to manually control the injection pressures.

Kyphoplasty is a modification of percutaneous vertebroplasty. Kyphoplasty involves a preliminary step that comprises the percutaneous placement of an inflatable balloon tamp in the vertebral body. Inflation of the balloon creates a cavity in the bone prior to cement injection. Further, the proponents of percutaneous kyphoplasty have suggested that high pressure balloon-tamp inflation can at least partially restore vertebral body height. In kyphoplasty, it has been proposed that PMMA can be injected at lower pressures into the collapsed vertebra since a cavity exists to receive the cement—which is not the case in conventional vertebroplasty.

The principal indications for any form of vertebroplasty are osteoporotic vertebral collapse with debilitating pain. Radiography and computed tomography must be performed in the days preceding treatment to determine the extent of vertebral collapse, the presence of epidural or foraminal stenosis caused by bone fragment retropulsion, the presence of cortical destruction or fracture and the visibility and degree of involvement of the pedicles. Leakage of PMMA during vertebroplasty can result in very serious complications including compression of adjacent structures that necessitate emergency decompressive surgery.

Leakage or extravasion of PMMA is a critical issue and can be divided into paravertebral leakage, venous infiltration, epidural leakage and intradiscal leakage. The exothermic reaction of PMMA carries potential catastrophic consequences if thermal damage were to extend to the dural sac, cord, and nerve roots. Surgical evacuation of leaked cement in the spinal canal has been reported. It has been found that leakage of PMMA is related to various clinical factors such as the vertebral compression pattern, and the extent of the cortical fracture, bone mineral density, the interval from injury to operation, the amount of PMMA injected and the location of the injector tip. In one recent study, close to 50% of vertebroplasty cases resulted in leakage of PMMA from the vertebral bodies. See Hyun-Woo Do et al, “The Analysis of Polymethylmethacrylate Leakage after Vertebroplasty for Vertebral Body Compression Fractures”, Jour. of Korean Neurosurg. Soc. Vol. 35, No. 5 (May 2004) pp. 478-82, (http://wwwjkns.or.kr/htm/abstract.asp?no=0042004086).

Another recent study was directed to the incidence of new VCFs adjacent to the vertebral bodies that were initially treated. Vertebroplasty patients often return with new pain caused by a new vertebral body fracture. Leakage of cement into an adjacent disc space during vertebroplasty increases the risk of a new fracture of adjacent vertebral bodies. See Am. J. Neuroradiol. 2004 February; 25(2): 175-80. The study found that 58% of vertebral bodies adjacent to a disc with cement leakage fractured during the follow-up period compared with 12% of vertebral bodies adjacent to a disc without cement leakage.

Another life-threatening complication of vertebroplasty is pulmonary embolism. See Bernhard, J. et al., “Asymptomatic diffuse pulmonary embolism caused by acrylic cement: an unusual complication of percutaneous vertebroplasty”, Ann. Rheum. Dis. 2003; 62:85-86. The vapors from PMMA preparation and injection are also cause for concern. See Kirby, B., et al., “Acute bronchospasm due to exposure to polymethylmethacrylate vapors during percutaneous vertebroplasty”, Am. J. Roentgenol. 2003; 180:543-544.

Another disadvantage of PMMA is its inability to undergo remodeling—and the inability to use the PMMA to deliver osteoinductive agents, growth factors, chemotherapeutic agents and the like. Yet another disadvantage of PMMA is the need to add radiopaque agents which lower its viscosity with unclear consequences on its long-term endurance.

In both higher pressure cement injection (vertebroplasty) and balloon-tamped cementing procedures (kyphoplasty), the methods do not provide for well controlled augmentation of vertebral body height. The direct injection of bone cement simply follows the path of least resistance within the fractured bone. The expansion of a balloon also applies compacting forces along lines of least resistance in the collapsed cancellous bone. Thus, the reduction of a vertebral compression fracture is not optimized or controlled in high pressure balloons as forces of balloon expansion occur in multiple directions.

In a kyphoplasty procedure, the physician often uses very high pressures (e.g., up to 200 or 300 psi) to inflate the balloon which first crushes and compacts cancellous bone. Expansion of the balloon under high pressures close to cortical bone can fracture the cortical bone, or cause regional damage to the cortical bone that can result in cortical bone necrosis. Such cortical bone damage is highly undesirable and results in weakened cortical endplates.

Kyphoplasty also does not provide a distraction mechanism capable of 100% vertebral height restoration. Further, the kyphoplasty balloons under very high pressure typically apply forces to vertebral endplates within a central region of the cortical bone that may be weak, rather than distributing forces over the endplate.

There is a general need to provide systems and methods for use in treatment of vertebral compression fractures that provide a greater degree of control over introduction of bone support material, and that provide better outcomes. Embodiments of the present invention meet one or more of the above needs, or other needs, and provide several other advantages in a novel and non-obvious manner.

### SUMMARY OF THE INVENTION

The invention provides a method of treating bone abnormalities including vertebral compression fractures, bone tumors and cysts, avascular necrosis of the femoral head and tibial plateau fractures. In an exemplary embodiment, the system of the invention provides small cross-section implant elements that can be introduced into targeted bone regions wherein the elements self-assemble into a higher modulus monolith. The implant elements have surface features that are configured for engaging one another to interlock the plurality of elements when subjected to compression. The implantable elements also can have selected open cell characteristics that are optimized for tissue ingrowth.

In one exemplary system and method, the cancellous bone in a fractured vertebra is accessed by boring into the damaged vertebral body. Thereafter, the elements are introduced into the targeted site through a small diameter introducer. The elements are configured for engaging one another, either by means of surface engaging features or by means of magnetic properties. After a selected volume of the elements are packed into bone to displace cancellous bone, additional volumes of elements will apply forces on the vertebral endplates to reduce the fracture.

### BRIEF DESCRIPTION OF THE DRAWINGS

In the following detailed description, similar reference numerals are used to depict like elements in the various figures.

FIG. 1 is a view of exemplary implant elements corresponding to the present invention in perspective and cut-away views.

FIG. 2 depicts small number of the elements of FIG. 1 after being compressed together, meshed and interlocked to form a portion of a monolith.

FIG. 3 is a view of a segment of a spine with a vertebral compression fracture that can be treated with the present invention, showing an introducer in a method of the invention.

FIG. 4A is a cross-sectional view of the vertebra and abnormality of FIG. 3 with a single treatment region therein.

FIG. 4B is a cross-sectional view of the vertebra and abnormality of FIG. 3 with a plurality of treatment regions therein.

FIG. 5 is a view of an alternate system that includes first spiked implant elements for irreversibly engaging second implant elements that comprise bodies of entangled or woven microfilaments.

FIG. 6 is a view of the first and second implant elements of FIG. 5 in the bore of an introducer.

FIG. 7 is a view of an alternative method of the invention wherein the implant elements have magnetic properties, and wherein magnetic forces cause self-assembly of the elements into a substantially solid monolith.

### DETAILED DESCRIPTION OF THE INVENTION

The present invention relates to bone implant systems that include a plurality of small-cross section elements that are configured with coupling properties or features for in-situ assembly in bone of a substantially solid implant body. The elements can be introduced into cancellous bone through a small diameter introducer sleeve. The implants are particularly adapted for supporting bone in treating vertebral compression fractures (VCFs). In several embodiments, the individual elements are reticulated or porous to allow for bone ingrowth.

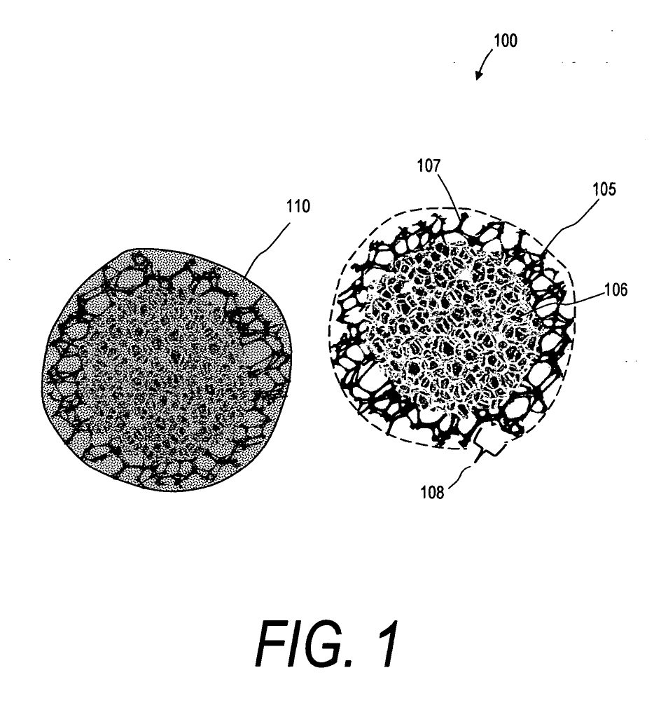

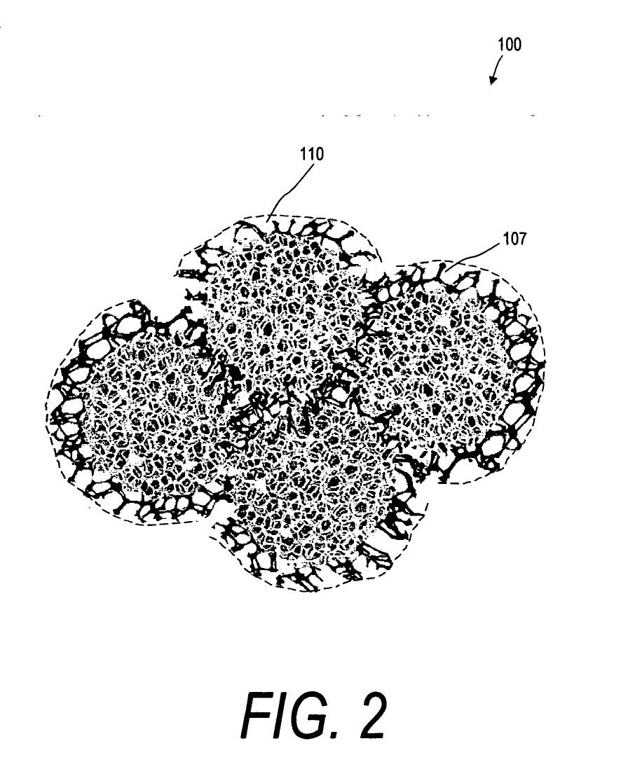

FIG. 1 illustrates a greatly enlarged view of an exemplary implant element 100 that comprises a reticulated metallic material. The term “reticulated” as used herein means having the appearance of, or functioning as, a wire-like network or substantially rigid network of struts or ligaments 105. The related term reticulate means resembling or forming a network. The terms reticulated and trabecular are used interchangeably herein to describe structures having ligaments 105 that bound open cells 106 in the interior of the element or structure (see FIG. 1). The elements 100 of FIGS. 1 and 2 are configured with surface ligament projections 107 that allow for irreversible, meshed interlocking of elements 100 upon a selected level of compressive forces. As can be seen in FIG. 1, the peripheral region 108 of an element 100 has generally radially-extending jagged ligaments 107 that are exposed. In the peripheral region 108, the non-radial ligament portions are removed. Further, the peripheral region can have ligament projections that are bendable to allow and enhance irreversible entanglement upon compression with an adjacent element. The elements 100 preferably have a mean cell cross section ranging from about 10 microns to 1000 microns, and more preferably from about 100 microns to 400 microns. The cell dimensions can be selected for enhancing tissue ingrowth. In any embodiment, the reticulated structure has a modulus ranging between 0.01 and 15 GPa, and more preferably between 0.10 and 10 GPa. Preferred materials for the elements 100 are titanium, tantalum or a magnesium alloy that is biodegradable following implantation. Numerous manners are available for fabricating such open-volume materials, for example, as offered by Porvair Advanced Materials, Inc., 700 Shepherd Street, Hendersonville N.C. 28792.

Such a reticulated structure 100 as in FIG. 1 is further defined by density-which describes the ligament volume as a percentage of a solid. In other words, the density defines the volume of material relative to the volume of open cells 106 in a monolith of the base material. As density of the ligaments increase with larger cross-sections and smaller cell dimensions, the elastic modulus of the material will increase. The cells of the interior of reticulated structure 100 (FIG. 1) also can have a mean cross section which is less than cells at surface region 108. In preferred embodiments, the cells are bounded by polyhedral faces, typically pentagonal or hexagonal, that are formed with five or six ligaments 105. In exemplary embodiments, as also represented in FIG. 1, the reticulated structure of elements has a density that ranges between 2% and 80%, and preferably ranges between 5% and 50%.

In FIGS. 1 and 2, it further can be seen that elements 100 have at least a surface layer of a polymeric composition 110. The polymeric composition 110 can be any material that yields under a selected level pressure, such as an open cell foam, or a brittle, fracturable polymer layer. The exemplary embodiment of FIGS. 1 and 2 shows the polymer 110 as infilling the interstices of the elements. In this embodiment, the elastomeric composition has an elastic modulus ranging between 5 MPa and 100 KPa. In another embodiment, the polymeric composition 110 is a thin sacrificial layer or shell gives way when compressed. In one embodiment, the polymer composition 110 is at least one of bioerodible, bioabsorbable or bioexcretable. The purpose of the polymer surface is to prevent meshing and interlocking of elements 100 when handling and for introduction purposes. Of particular interest, a selected level of compression against surface 108 of the elements will cause the polymer to deform, fracture or collapse to expose ligament projections 107 which comprise ligament portions at surface 108. For purposes of description, if one were to roll elements 100 between thumb and fingers, the elements would resemble pebbles. However, if one were to apply a selected high level of compression to multiple elements 100 held together, the surfaces give way causing the jagged ligament projections 107 to penetrate into adjacent elements in an irreversible Velcro-like manner to create a non-deformable assembly.

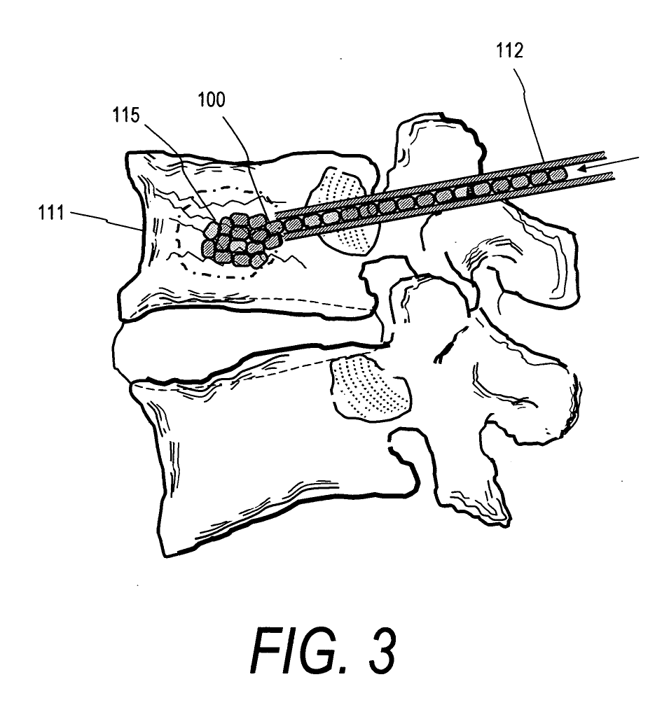

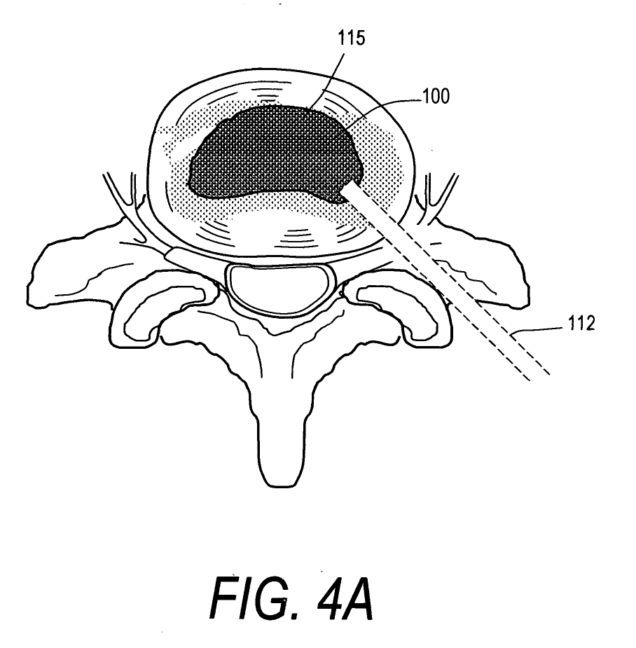

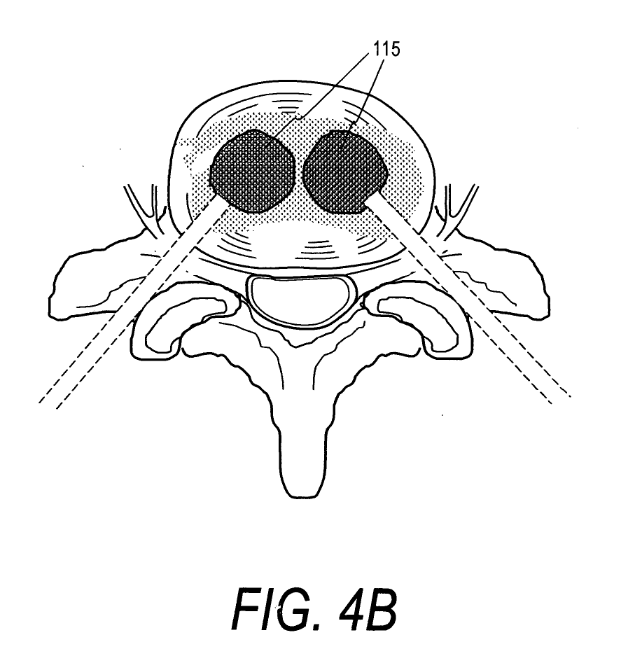

FIG. 2 illustrates several implant elements 100 meshed and interlocked. It can be understood that a large number of elements can be implanted and meshed as illustrated in FIG. 3 to support a bone. In one preferred embodiment as depicted in FIGS. 1 and 2, the implant elements 100 can be round, cubic, polygonal, or irregularly shaped and also may be elongated or tubular. In any embodiment, the cross section across a minor axis is less that about 5 mm and preferably less that about 3 mm. In FIG. 3, vertebra 111 has an abnormality such as a compression fracture. An introducer 112 with a diameter ranging from about 2 mm to 5 mm in diameter is shown in a transpedicular access to the interior of the vertebra. As can be seen in FIG. 3, a plurality of elements 100 are shown being ejected from introducer 112 to occupy and displace cancellous bone with a monolithic, irreversibly locked-together implant 115. The elements 100 are ejected from the introducer by a pusher mechanism, which preferably is motor driven and designed to provide high injection forces. FIGS. 4A and 4B indicate that the treatment site in a vertebra can be a single region or multiple spaced apart regions.

In general, the projecting surface features for meshing and interlocking elements 100 include variations in the surface density of ligaments of a reticulated material, or variations in the percentage of radial vs. non-radial ligaments. The projecting features for meshing or interlocking elements 100 also can include wire-like elements independent of the ligaments of the reticulated structure, for example, wire-like elements of a shape memory alloy, a ductile metal or a polymer that function as at least one of spikes, barbs, hooks, protuberances and burrs.

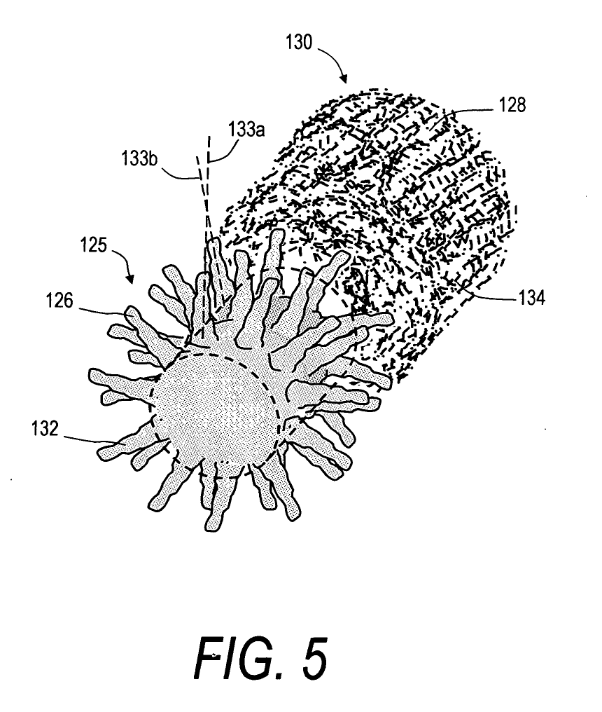

Now turning to FIG. 5, an alternative implant system uses a plurality of first implant elements or “spiked” elements 125 which include a plurality of projecting features or spikes 126 that are adapted penetrate into, or engage, cooperating projection-receiving features 128 of a second cooperating implant element 130. In this embodiment, the projecting features or spikes 126 are carried in each element 125, and comprise a plurality of Velcro-like projections with articulated (non-smooth or non-linear) surfaces 132 for engaging structure into which the projections 126 are penetrated—which thus functions similar to Velcro. The axes (133a, 133b etc.) of the various projections are also preferably varied. The second cooperating elements 130 as depicted in FIG. 5 comprise a fabrication of microfilaments 134 formed into a wad, sphere-like body or cylindrical body that allows for its introduction together with first elements 125. The microfilaments 134 can be any suitable biocompatible material such as carbon fiber, stainless steel, titanium, magnesium alloys and the like. The microfilament fabrication can comprise a structure of entangled filaments 134 similar to stainless steel wool, except with a bonded together portions or welds within the steel wool to make it impossible to disentangle. Thus, when the Velcro-like projections 126 penetrate into the entangled filaments 134, the coupling is very strong. The structure of filaments 134 can also comprise woven fabrications, knit fabrications and braided fabrications that would function in a similar manner to couple together a plurality of first and second elements 125 and 130 for in-situ assembly of a substantially solid monolith.

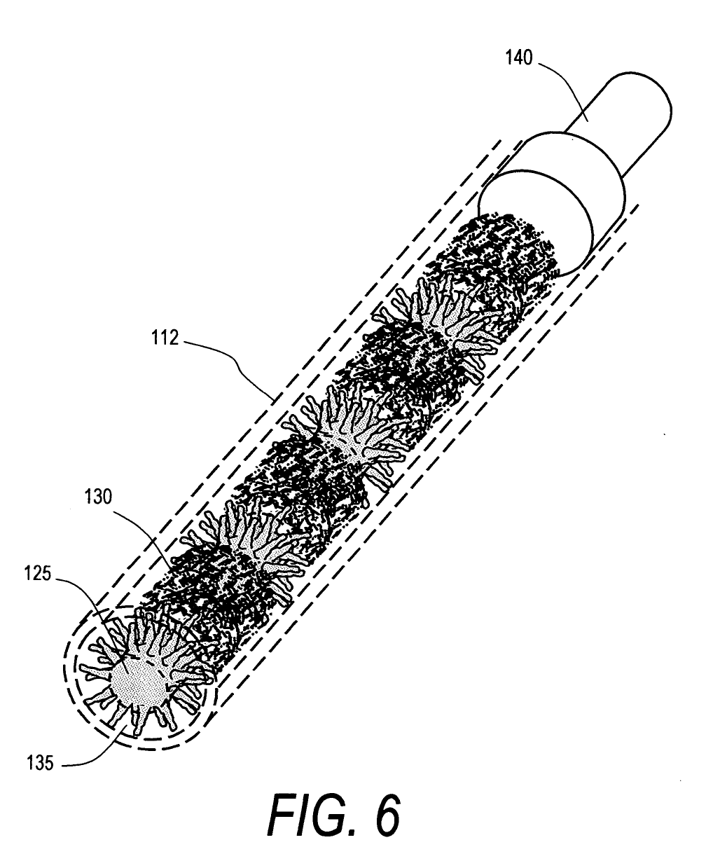

FIG. 6 illustrates an assembly of first and second implant elements 125 and 130 in bore 135 of introducer 112 wherein a pusher mechanism 140 is used to push the elements outwardly into a targeted interior region of a bone. The pusher mechanism 140 is preferably motor-driven by a screw mechanism.

In another similar embodiment, each of the “spiked” elements 125 as in FIG. 5 can be configured with a microfilament fabrication of entangled or woven filaments 134 in and around the projections 126. The filaments 134 are thus crushable to receive and grip the spikes 126 of another element 125 when a group of such elements are crushed together under substantial force (cf. FIG. 2). The filaments 134 further can be infused with a composition such as a polymer that bonds the filaments together but yields or sacrifices under mechanical forces as when spikes 126 from another element are pressed into the filament structure. In use, a plurality of linearly-stacked elements 125 can be pushed from bore 135 of an introducer as in FIG. 6 to infill an interior of an abnormal bone. The plurality of elements will thus self-assemble and irreversible couple to create a monolithic implant.

Referring back to FIG. 5, in one embodiment, the implant elements can have rough-surfaced projecting features of a metal with an extension dimension ranging from about 0.1 mm to 2.0 mm, and preferably from about 0.2 mm to 1.0 mm. The surface features can be fabricated in an automated manner by a novel electron beam system that generates ordered protrusions in metal surfaces for commercial applications. The system was developed for mechanically bonding metal structure to carbon fiber composites wherein the metal penetrating elements in aircraft composites and other industrial applications. The electron beam system is being commercialized under trade names “Surfi-Sculpt” and “Comeld” by TWI Ltd., Granta Park, Great Abington, Cambridge, CB1 6AL, UK (see, e.g., www.twi.co.uk and http://www.camvaceng.co.uk/surfisculpt.asp, which are incorporated herein by this reference).

The spiked elements 125 of the invention (FIG. 5) also can be fabricated by other suitable means for fabrication of projections and spikes, such as metal injection molding (MIM), polymer injection molding followed by metal plating, casting, forming, stamping, and the like to create projecting features in multiple surfaces of an implant element.

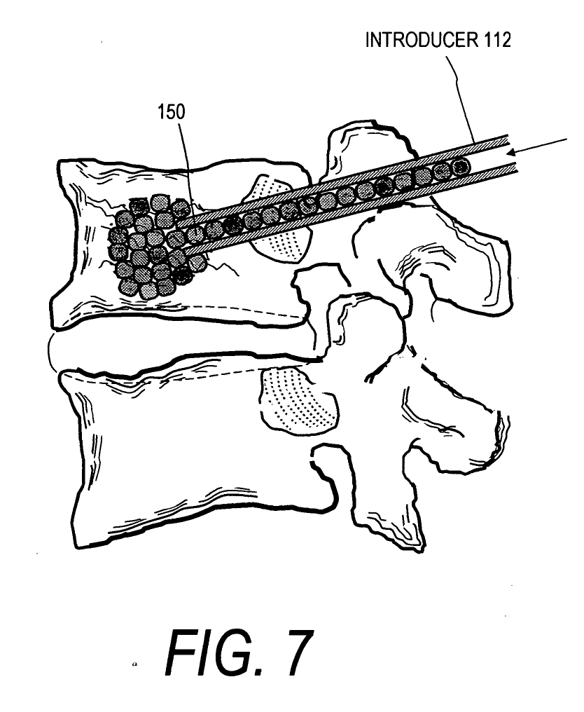

FIG. 7 illustrates another system and method corresponding to the invention wherein the implant elements 150 comprise magnetic elements that engage one another to provide a substantially solid monolith by means of magnetic properties of elements. The magnetic elements can be any suitable biocompatible materials, and can be rare earth magnetic materials that exhibit strong magnetic properties. For example, the elements can be rare earth Neodymium-Iron-Boron (NIB) magnets or the like. The elements 150 can be spherical, faceted, polygonal or elongated and have a mean cross section ranging between about 0.5 mm and 5 mm for introduction by a pusher mechanism through an introducer as depicted in FIG. 7. In use, the magnetic elements will aggregate about the end of introducer 112 and self-adjust as more and more elements accumulate and engage one another. The aggregation of magnetic elements will tend to form a spherical shape which will displace cancellous bone and apply forces on the cortical endplates to reduce the fracture. The scope of the invention further includes introducing an in-situ hardenable bone cement (e.g., PMMA) into the elements to further lock together the elements 150 into a monolithic implant. In another embodiment, the method of the invention includes introducing varied sizes of magnetic elements. For example, a first volume of small diameter magnetic elements 150 can be injected followed by additional volumes of at least one larger diameter elements. Thereafter, a flowable and curable bone cement is introduced into the interior of the volume of magnetic elements 150 wherein the elements adjust to form a barrier at the surface to the infill volume to prevent extravasion of the bone cement. In this method of the invention, the infill materials self-organize in-situ into an expandable surface that can substantially contain a bone cement to prevent flow of the cement in undesired directions. The elements also assembly in-situ into a surface that can expand to apply forces to cortical endplates to at least partly restore vertebral height.

As can be understood from FIGS. 3, 6 and 7, preferred methods of the invention utilize an introducer sleeve 112 for inserting the implant elements by means of a pusher mechanism. In another embodiment, the pusher mechanism and/or introducer is coupled to an energy source for vibrating the distal end of the assembly at low frequencies, for example upwards of 10 Hz, or alternatively at ultrasound frequencies as is known in the art. Thus, the introducer tip can be used to fracture cancellous bone to create a space contemporaneously with the ejection of each implant element 125. Further, the tip can be deflectable as is known in the art to allow the implantation and self-assembly of a highly irregular-shaped rigid porous monolith—as is sometimes needed in a treatment of a vertebral compression fracture.

Of particular interest, the system also may be used in a prophylactic manner with small introducers, for example, to provide bone support in vertebrae of patients in advance of compression fracture.

In another embodiment, the invention encompasses orthopedic implants configured for implantation within or proximate to bones that include at least one body formed at least partially of a biodegradable magnesium alloy. The method of the invention includes treating an orthopedic abnormality by (a) introducing a bone support into or proximate to a bone wherein the bone support includes a biodegradable magnesium alloy, and (b) allowing the magnesium alloy to biodegrade thereby creating space for tissue ingrowth. This method encompasses the use of such bone supports in the form of at least one of implant elements, fill materials, cages, screws, rods, and stents.

In any embodiment, the implant elements further can carry a radiopaque composition if the material of the implant itself is not radiovisible.

In any embodiment, the implant elements further can carry any of the following: antibiotics, cortical bone material, synthetic cortical replacement material, demineralized bone material, autograft and allograft materials. The implant body also can include drugs and agents for inducing bone growth, such as bone morphogenic protein (BMP). The implants can carry the pharmacological agents for immediate or timed release.

The above description of the invention is intended to be illustrative and not exhaustive. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

What is claimed:

1. A bone implant system comprising a plurality of elements configured with properties for in-situ coupling of the elements to form a substantially non-deformable implant body.

2. The bone implant system of claim 1 wherein the plurality of elements are configured with at least one of projecting features or projection-gripping features.

3. The bone implant system of claim 1 wherein the plurality of elements are configured with projecting features and projection-gripping features intermediate said projecting features.

4. The bone implant system of claim 2 wherein the elements are at least partly fabricated of a biodegradable magnesium alloy.

5. The bone implant system of claim 2 wherein the projection-gripping features comprise openings in a microfilament fabrication.

6. The bone implant system of claim 4 wherein the fabrication is selected from the group of entangled filament fabrications, woven fabrications, knit fabrications and braided fabrications.

7. The bone implant system of claim 4 wherein the fabrication includes carbon fiber microfilaments.

8. The bone implant system of claim 4 wherein the fabrication includes metal microfilaments.

9. The bone implant system of claim 7 wherein microfilaments are at least one of stainless steel, titanium and a magnesium alloy.

10. The bone implant system of claim 1 wherein at least a portion of the elements are of a reticulated material.

11. The bone implant system of claim 1 wherein at least a portion of the elements are configured with magnetic properties.

12. The bone implant system of claim 1 wherein the elements configured with magnetic properties.

13. The bone implant system of claim 12 wherein the elements configured with magnetic properties have varied cross-sections.

14. A method for treating an abnormality in a bone comprising (a) introducing into a bone a plurality of elements configured with surface projections and projection-gripping features, and (b) causing the surface features to irreversibly interlock amongst the elements thereby forming a substantially solid monolith.

15. The method of claim 14 wherein the abnormality is a fracture in a vertebra.

16. The method of claim 14 wherein steps (a) and (b) displace cancellous bone.

17. The method of claim 14 wherein steps (a) and (b) move cortical bone.

18. A method for treating an abnormality in a bone comprising introducing into a bone a plurality of elements configured with magnetic properties wherein the magnetic properties cause the plurality of element to self-assemble into a bone support structure.

19. The method of claim 18 wherein the elements configured with magnetic properties are at least one of spherical, polygonal, faceted or elongated.

20. The method of claim 18 further comprising the step of introducing a bone cement into the plurality of elements.

21. A method for treating an abnormality in a bone comprising (a) introducing a bone support into or proximate to a bone, the bone support including a biodegradable magnesium alloy, and (b) allowing the magnesium alloy to biodegrade thereby creating space for tissue ingrowth.

22. The method of claim 21 wherein step (a) includes introducing a bone support in the form of at least one of implant elements, fill materials, cages, screws, rods, and stents.

23. An orthopedic implant configured for implantation in or proximate a bone comprising at least one body including a biodegradable magnesium alloy.
